(19) United States
(12) Patent Application Publication
(10) Pub. No.: US 2022/0336061 A1
(43) Pub. Date: Oct. 20, 2022

(54) METHOD AND SYSTEM FOR IMPROVING PATIENT PRE-SCREENING FOR RECRUITMENT IN CLINICALS TRIALS

(71) Applicant: CLINICAL TRIALS MOBILE APPLICATION, Asnières-sur-Seine (FR)

(72) Inventors: Clare MACKENZIE, Paris (FR); Norman SABBAH, Paris (FR)

(73) Assignee: CLINICAL TRIALS MOBILE APPLICATION, Asnières-sur-Seine (FR)

(21) Appl. No.: 17/856,106

(22) Filed: Jul. 1, 2022

Related U.S. Application Data
(63) Continuation-in-part of application No. PCT/EP2021/050002, filed on Jan. 3, 2021.
(60) Provisional application No. 62/956,792, filed on Jan. 3, 2020.

(51) Int. Cl. G16H 10/20

(52) U.S. Cl. CPC G16H 10/20

(57) ABSTRACT

A method and system for pre -screening of a patient with regard to eligibility criteria of at least one clinical trial, preferably during a medical team-patient interaction, including: an interface that displays a list of medical-team-oriented questions reflecting at least one, preferably 2 to 15, preferably 5 to 10, clinical trial eligibility criteria; registers the patient's response, the response being collected by the medical team, so that they are real-time answers at the time of pre-screening; if the response meets the eligibility criteria of at least 1-5 clinical trials, displays a list of the clinical trials; and upon selection of one clinical trial by the physician in the list, upon collection of some patient data and upon agreement of the patient, sends to the clinical research team in charge of the selected clinical trial, a message notifying the pre-screening of the patient.

### FIELD

The present invention relates to the field of healthcare informatics, especially to the handling of information and communication technologies for detection and recruitment of patients in clinical trials. More especially, this invention relates to an efficient way of both finding out the patients that meet the eligibility criteria of a clinical trial, and prescreen them for enhancing the chances of success of enrolment into the trial.

### BACKGROUND

Many barriers contribute to non-enrollment in clinical trials, including patient, physician, protocol, and regulatory barriers. At the protocol level, eligibility criteria have become a large roadblock to clinical trial recruitment.

Several attempts to solve this problem have been made previously, as described for example in: international patent application WO2015/018927 (Y.Bouhnik); U.S. Pat. No. 7,904,313 (Quintiles) and U.S. Pat. No. 10,366,780 (Elligo).

For instance, in the context of inflammatory bowel diseases (IBD), in the last 20 years (1998-2018) the average recruitment rate dropped from 0.32 to 0.13 patients per site per month in moderate-to-severe ulcerative colitis, and from 0.65 to 0.1 patients per site per month in moderate to severe Crohn's disease (M. S. Harris et al. (2019), “Competition for Clinical Trials in Inflammatory Bowel Diseases”, Gastroenterology, 157(6): 1457-1461).

Due to under-recruitment, clinical trials often need to be extended, in an effort to reach the required number of participants; suspended; terminated; or withdrawn. This has several negative consequences such as waste of funds; reduction of the quality of care delivered to the trial participants; and delay for patients to access therapeutic innovation, when the trial is completed despite under-recruitment, failure to reach an acceptable level of statistical power.

In addition to low recruitment rates, maintaining an acceptable number of patients after recruitment is also a major challenge.

Furthermore, current pre-screening procedures are time consuming, and valuable time and effort of the physician is lost searching clinical trials that are appropriate for his/her patients.

According to the Applicant, the reason why the prior art does not resolve this issue may be explained by the following: (1) the patient are poorly selected because the patient's regular medical environment (physicians, specialists) is not easily made aware of the clinical trials; or is not put in a position where they can quickly understand the eligibility criteria of clinical trials; or cannot easily assess a possible match with a patient they know; or (2) the patients are contacted with surveys that are too long and burdensome, and in some way alarming or frightening or do not really make sense to them.

Eligibility criteria in clinical trials may show a real complexity. Ross J et al described it as of 2010, in a publication entitled “Analysis of eligibility criteria complexity in clinical trials” (Ross J et al AMIA Summits Transl Sci Proc. 2010; 2010:46-50). In this article, the eligibility criteria were classified by their complexity, semantic patterns, clinical content, and data sources. The analyses revealed significant semantic and clinical content variability. The article concludes that 85% of the eligibility criteria have significant semantic complexity.

Also, medical teams are busy and have very limited time, and cannot afford dissecting the eligibility criteria on one hand and get personal information from the patient, asked in some of the eligibility criteria, on the other hand. On the other hand, fully relying on patient database means that information may not be accurate or up-to-date.

It has to be highlighted that each clinical trial shows a number of complex and diverse eligibility criteria (inclusion criteria, and also exclusion criteria), constituting a huge workflow to deal with.

There is therefore a need to find methods and systems that manage the number and complexity of eligibility criteria for each clinical trial, but also for several clinical trials in a row, for a specific condition, actually ending up with an improved enrolment and increase the number of patients that could be enrolled. This method comprises a means, i.e. a questionnaire with a limited number of questions, the questions being easy to answer, for facilitating identification and pre-screening of patients for further enrolment in clinical trials, in a very rapid time frame, despite the complexity of the eligibility criteria, and with the insurance to gather up-to-date information from the patient, together with an agreement of the patient regarding the pre-screening, and the fact that the pre-screening and personal data will be forwarded to the investigation center.

### SUMMARY

This invention thus relates to a system for pre-screening a patient for potential inclusion in a clinical trial during a medical team-patient interaction comprising a computer platform including relevant questions reflecting clinical trial eligibility criteria, where the questions are designed in number and in content for a few-minutes pre-screening of the patients with regard to the clinical trial eligibility criteria.

In a first aspect, the system of the present invention comprises an interface for pre-screening of a patient with regard to eligibility criteria of at least one clinical trial that:
displays a list of medical-team-oriented questions reflecting at least one, preferably 2 to 15, preferably 5 to 10, clinical trial eligibility criteria,
receive the patient's response, the response being collected by the medical team, so that they are real-time answers at the time of pre-screening
if the response meets the eligibility criteria of at least 1-5 clinical trials, displays a list of said clinical trials,
upon selection of one clinical trial by the physician in the list, upon collection of some patient data and upon agreement of the patient, sends to the clinical research team in charge of the selected clinical trial, a message notifying the pre-screening of the patient.

The medical team may be composed of physicians, study coordinator, or clinical research assistants.

An advantage of the invention is to increase the number of identified and pre-screened patient.

In one embodiment, the patient's response may be registered in a storage medium.

In one embodiment, the interface may be further configured to record the medical-team-oriented questions in the form of a decision tree (FIG. 4).

In one embodiment, in order to determine if the response meets the clinical trial eligibility criteria, the processor is configured to select relevant inclusion and exclusion criteria for one or more clinical trial, using a machine learning algorithm trained on a remote database (such as clinicaltrials.gov). In one embodiment, the processor selects relevant eligibility criteria for more than one clinical trial, ie 2 to 15 clinical trials, preferably directed to the same medical condition.

In one embodiment, the system according to the present invention comprises a database with a list of clinical trial eligibility criteria and a list of clinical trials, wherein each clinical trial eligibility criterion is linked to at least one clinical trial, and each clinical trial is linked to a clinical research team. Each data of the said database may be entered by a user, or retrieved automatically from a remote database hosted in a remote server comprising data related to privately and publicly funded clinical studies conducted around the world (e.g.: clinicaltrials.gov database).

In one embodiment, the system comprises an encryption module for generating encrypted data and/or pseudonymized and/or anonymized patient data. For instance, the system may comprise an encryption, pseudonymization or anonymization software or a connection to such software.

In one embodiment, the medical-team-oriented questions are displayed as checkbox questions, meaning that the response to the question is provided by checking one or several boxes. In one embodiment, the medical-team-oriented questions are binary (such as for example yes/no), multiple-choice questions, and/or numeric questions (i.e. the answer is a number). In one embodiment, the medical-team-oriented questions are closed-ended questions. In one embodiment, the medical-team-oriented questions are multiple-choice question triggering 2 to 5 possible responses; the possible responses may be displayed in a drop-down list.

In one embodiment, the medical-team-oriented questions are mastered from the original clinical trial criteria, and drafted to achieve a complete and quick understanding by a physician of the criteria, so that he can rapidly (i.e. in 10 seconds to 3 minutes) get the response and real-time information from the patient.; a set of questions achieving this goal is referred to, in this invention, as a straightforward question. In one embodiment, a straightforward question has a number of words ranging from 3 to 35 words, preferably from 4 to 15 words.

In one embodiment, the time for successfully filling up the preliminary enrolment process last from 5 seconds to 5 minutes, preferably 1 to 3 minutes.

The medical-team-oriented questions are drafted by a team of experts that transforms the eligibility criteria into straightforward set of discriminative questions/proposed responses, so that the fulfilment of the set of questions directs to less than 5 clinical trials.

In one embodiment, once the questions have been drafted by a team of experts, the questions may be simply recorded in the form of a decision tree in the system. Said recording step may be advantageously monitored or supervised by people with no or limited medical skills (FIG. 4). Questions may be binary questions (such as for example yes/no), multiple-choice questions, and/or numeric questions. The system is easily scalable if any other question types are needed for the implementation of the questionnaire.

In one embodiment, the system is configured by investigation center. This means that the system displays only the protocols that are open in this investigation center.

By investigation center is meant a medical department having at least one valid contract with a sponsor for an on-going clinical trial.

In one embodiment, the questions are gathered on a single screen. In one embodiment, the questions are separated in several question lists, each list being displayed on one screen. In this embodiment, the screens may be successive. In one embodiment, when one response is contrary to the expected response for fulfilling one criteria of a clinical trial, the interface displays a non-eligibility message to the medical team, or proposes a switch to another clinical trial.

In one embodiment, the medical team-patient interaction is a medical face-to-face or virtual or telephone appointment.

In one embodiment, the patient data includes name, surname, and telephone number of the patient. The patient data may further include an e-mail address. The patient data may further include a localization data, such as for example a zip code, state name, town name, and/or home address. The patient data may further include date of birth, place of birth.

In one embodiment, the system is located on a physical or virtual server. The server may be connected to a computer network. In one embodiment, the computer network is hosted on a cloud.

In one embodiment, the system is located on a physical or virtual server. The server may be connected to a computer network. In one embodiment, the computer network is hosted on a cloud.

In one embodiment, the system comprises: 1

at least one pre-screening module (patient eligibility module—accessible to the medical team—in one embodiment, the pre-screening module can be accessed by the clinical research assistant or study coordinator),
at least one message sender module,
at least one patient follow-up module for the follow-up of the patients (patient follow-up module—accessible to the investigator team),
at least one module with the clinical trials open in the investigator center with the full list of eligibility criteria including non limitatively inclusion/exclusion criteria (clinical trial module—with items accessible to the medical team, and items accessible to investigator team),
at least one piece of key information on the clinical trial goals or specificities,
at least one database of straightforward questions and related responses,
at least one users' database with a Software as a Service (SAAS) mode for managing users' access,
at least one patient data form,
at least one encryption, pseudonymization or anonymization software or connection to such software, and/or
at least one message sender or connection to any messenger system, to forward the pre-screening results to the investigation center.

In one embodiment, the open clinical trials are all and any clinical trials that are recruiting in any country of the world, and for all and any therapeutic area.

In one embodiment, the system may display a full list of clinical trials, or a full list of original criteria for a clinical trial.

In one embodiment, the open clinical trials are related to the treatment of inflammatory or auto-immune diseases, including non limitatively IBD, UC or CD. In one embodiment, the physician is a specialist of inflammatory or autoimmune diseases.

In one embodiment, the open clinical trials are related to the treatment of cancer. In one embodiment, the physician is an oncologist.

In one embodiment, the interface is operated by a computer system comprising a software application automatically computing information from the clinical trials database to select, sort, compare, read, or write records from the said database.

In one embodiment, the system is connected to or includes a security layer including an encryption and/or pseudonymization and/or anonymization software, so that the patient data and agreement are protected, i.e. encrypted, pseudonymized and/or anonymized prior to being sent to the clinical research team. In one embodiment, the patient data are encrypted with an AES -256 symmetric encryption algorithm.

In a second aspect, the invention relates to a method and system for pre-screening of a patient with regard to eligibility criteria of at least one clinical trial, the method ensuring that the patient data are current, and being implemented preferably but not limitatively during a medical team-patient interaction.

In one embodiment, the system comprises:
a prescreening module having at least one interface that:
displays a list of medical-team-oriented questions reflecting at least one, preferably 2 to 15, preferably 5 to 10, clinical trial eligibility criteria,
registers the patient's response, the response being collected by the medical team, so that they are real-time answers at the time of pre-screening
if the response meets the eligibility criteria of at least 1-5 clinical trials, displays a list of said clinical trials,
upon selection by the physician of one clinical trial in the list, displays a list of 1-5 summarized inclusion criteria and 0-5 summarized exclusion criteria for the selected clinical trial;
   registers the response to the list of summarized inclusion/exclusion criteria,
   if the response meets the inclusion and exclusion criteria: informs the physician that the protocol of the clinical trial seems to be compatible with the patient,
   offers the possibility of matching with criteria of other clinical trials, while keeping in memory all the already responded criteria,
   and asks for (i) collection of patient data that are further encrypted and anonymized in a security layer and (ii) collection of the patient agreement to forward the pre-screening result and anonymized data, to the investigation center . . .
   sends from a message sender to the clinical research team, a message notifying the pre-screening results, together with physician information.

The notifying message may comprise a patient code. In order to find personal data of the patient from the patient code that is in the notification, the research team can connect to a “site and patient management” module integrated in the present system.

The notification is advantageous in that it ensures the coordination within the clinical research team, and between the medical team and the clinical research team. The notification also helps the appointment to be made, as the research team may early reserve slots in the agenda for patient appointment.

In a third aspect, the system further comprises a patient interface.

The present invention also relates to a computer-implemented method for pre-screening a patient for potential inclusion in a clinical trial, preferably during a medical team-patient interaction.

In one embodiment, the method of the invention is implemented by a system as described hereabove.

In one embodiment, the method for pre-screening of a patient with regard to eligibility criteria of at least one clinical trial comprises:
displaying a list of medical-team-oriented questions reflecting at least one, preferably 2 to 15, preferably 5 to 10, clinical trial eligibility criteria;
receiving, as input, the patient's response collected by a medical team member, so as to obtain the patient's real-time answers at the time of pre-screening;
if the response meets the eligibility criteria of at least 1-5 clinical trials, displaying a list of said clinical trials;
receiving, as input, the selection of one clinical trial in the list of said clinical trials,
collecting patient data and patient agreement;
sending to the clinical research team in charge of the selected clinical trial, a message notifying the pre-screening of the patient.

In one embodiment, the list of medical-team-oriented questions is displayed on a limited number of screens, preferably on one screen.

In one embodiment, the medical team-patient interaction is a medical face-to-face or virtual appointment.

In one embodiment, the method of the invention comprises a preliminary step, where the patient connects to a patient interface, and answers a patient's questionnaire containing patient-oriented questions only. Upon registration of the response to the questions, the patient interface displays a number of matching studies. A second set of questions, that can be responded to in interaction patient-coordinator. The patient or the patient with the help of the coordinator may choose a clinical trial and investigation center.

The coordinator can be a physician, or a specialist of clinical trials, especially those present on the system. His role is to further assess if the patient may be successfully pre-screened.

When the patient accepts to be contacted by a coordinator, they discuss the possible prescreening of the patient, then, the pre-screening results shall be sent to the investigation center.

When the patient is on his own, the system provides the investigation center names and location, and information on the clinical trial, and provides a registration system for keeping a record of all the data, responses etc. . . . already addressed by the patient. The registration system may be a QR code.

In one embodiment, the prescreening results might be discussed in an interaction patient-physician having access to the medical-team interface.

In one embodiment, the system provides the patient with all the open clinical trial straightforward questions, whatever the investigation center.

Advantageously, the system may comprise a geolocalization module for matching the patient location with the closest investigation center, where a pre-screened patient could be care of.

In a fourth aspect of the invention, the data of the patient prescreening are aggregated for further uses, such as analytics, improvement processes, feasibility studies or data trading.

In one embodiment, some or all of the data of the pre-screenings, without any personal data, are aggregated and made accessible, for information to Sponsor (i.e. Sponsor of the clinical trial for which the aggregated pre-screenings was carried out).

In one embodiment, some aggregated pre-screenings are made accessible to a contract research organization, for research needs.

In one embodiment, some aggregated pre-screenings are made accessible to the Investigation Center.

In one embodiment, aggregated pre-screenings are used for designing possible corrective actions in order to improve pre-screening and increase patient recruitment.

In one embodiment, some or all of the data of the pre-screenings, from various investigation centers are aggregated, concatenated and compared with the criteria of a future study of a sponsor in order to recommend/select the most appropriate centers to the sponsor (feasibility study).

In one embodiment, some or all of the data of the pre-screenings are aggregated, concatenated, in order to define and analyze patient populations or define patient population patterns, for example but not limitatively to analyze patient that are enrolled or dismissed, after having pre-screened, or to analyze if and how patients actually meet or actually do not meet some of the criteria. An advantage of this, is to enhance criteria compatibility to patient populations.

The present method also relates to a computer program comprising program code instructions which, when executed by a computer, cause the computer to carry out the method according to any one of the embodiments described hereabove.

The present method also relates a non-volatile computer storage medium comprising instructions which, when executed by a computer, cause the computer to carry out the method according to any one of the embodiments described hereabove.

Definitions

In the present invention, the following terms have the following meanings:
“CT” refers to Clinical trials, i.e. scientific studies in human or animal medical therapy to evaluate the efficiency and safety of a diagnostic method or treatment or any research project that assigns human or animal subjects to intervention and/or comparison groups to study the cause and effect relationship between a medical intervention and the evolution of a state of health.
“IBD”: refers to Inflammatory bowel diseases.
“UC”: refers to Ulcerative colitis.
“CD”: refers to Crohn's disease
“ClinicalTrials.gov”: refers to the database of privately and publicly funded clinical studies conducted around the world.
“Medical-team interface”: refers to the interface displaying the straightforward questions and proposed response.
“Patient interface”: refers to an interface displaying patient-oriented questions
“Medical team” refers to the healthcare workers responsible of providing care specific to a patient's medical needs, such as for instance investigator physicians, non-investigator physicians, clinical research assistants, study coordinators; wherein each member of the medical team is further entitled to perform patient pre-screening. 1

“Clinical research team” refers to the healthcare workers, such as investigator physician, clinical research assistants, study coordinators, research nurses, data coordinator, in charge of conducting a clinical trial.
“Medical-team-oriented questions”: means a set of questions readily understood by the physician, where at least one question may require the expertise of a physician, or may need to be conveyed to the patient with a short explanation from the physician.
“Patient-oriented questions”: means a set of questions readily understood by the patient, that requires no medical expertise.
“Pre-screening a patient” means detecting a patient, i.e., identifying that a patient is potentially eligible to a clinical trial.
“Pre-screened patient” refers to a patient the questionnaire whose responses to the questions meet the major criteria of a clinical trial, but prior to his/her informed consent.
“Relevant inclusion and exclusion criteria” refer to the clinical trial criteria that are considered as major, i.e are relevant in the frame of the pre-screening phase.
“Screened patient” refers to a patient that has competed pre-screening and provided informed consent.
Questionnaire: list of questions created according to the present invention, based on the eligibility criteria of at least one clinical trial; the questionnaire may be based on whole or part of eligibility criteria; in one embodiment, a selection of the major eligibility criteria is carried out prior to creating the questionnaire. The selection may be processed manually or be a computer-implemented first selection. It can be followed by a computer-implemented second selection prior to creating the questionnaire. The selected eligibility criteria (inclusion criteria and/or exclusion criteria) are herein referred to as the relevant eligibility criteria.
“Recruited patient” refers to a patient that has been enrolled and/or is randomized within the clinical trial.
“Recruitment rate” refers to the number of recruited patients per site per month, divided by the number of pre-screened patients per site per month.

### BRIEF DESCRIPTION OF THE DRAWINGS

FIG. 1 depicts a process flowchart with a one-step survey for a medical team-patient interaction during a medical appointment where the physician proposes to the patient a recruitment into a clinical trial.

FIG. 2 depicts a process flowchart with a two-steps survey for a medical team-patient interaction during a medical appointment where the physician proposes to the patient a recruitment into a clinical trial.

FIG. 3 depicts a process flowchart with a preliminary patient questionnaire, followed either a process of FIG. 1 or a process of FIG. 2, or a coordinator interaction and direct enrolment by the research team.

FIG. 4 depicts a process flowchart describing the steps leading to the questionnaire development.

### DETAILED DESCRIPTION

I. The system

The present invention relates to a system for pre-screening of a patient with regard to eligibility criteria of at least one clinical trial; the system comprising an interface, a transmission system and a processor. The system may further comprise a questionnaire creation module. The eligibility criteria of a clinical trial may be selected, in order to sort the major eligibility criteria (i.e. criteria relevant for pre-screening a patient). In one embodiment, some criteria may be selected among those published in the clinical trials to produce the questionnaire creation module with a first selection of criteria.

Preferably, the system is configured for pre-screening the patient during a medical team-patient interaction.

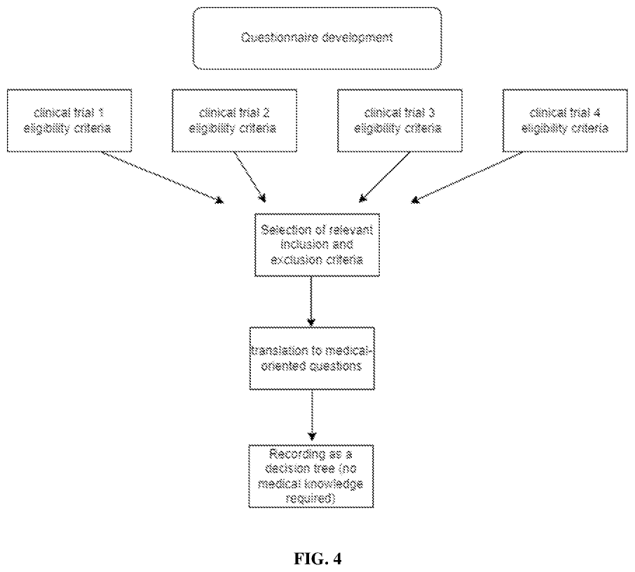

The questionnaire creation module may be configured to record medical-team-oriented questions in the form of a decision tree (FIG. 4). Such questions may advantageously be recorded by anyone without medical knowledge. Questions may be binary (such as for example yes/no), multiple-choice questions and/or numeric questions. The system is easily scalable if any other question types are needed for the implementation of the questionnaire.

In the present invention, the interface is configured to:
display a list of medical-team-oriented questions reflecting at least one, preferably 2 to 15, preferably 5 to 10, clinical trial eligibility criteria;
register the patient's response, the response being collected by the medical team, so that they are real-time answers at the time of pre-screening;
if the response meets the eligibility criteria of at least 1-5 clinical trials, displays a list of said clinical trials;
receive a selection of one clinical trial in the list by a member of the medical team, data of the patient, and agreement of the patient;
the processor is configured to determine if the response meets the eligibility criteria reflected by the medical-team-oriented questions; and the transmission system is configured to send to the clinical research team in charge of the selected clinical trial, a message notifying the pre-screening of the patient.

In one embodiment, the eligibility criteria verification may be done both based on studies stored in the system and on other center's studies based of the information provided on a remote database (e.g.: clinicaltrials.gov). In one embodiment, the processor is further configured to check eligibility (i.e. determine if the response meets the clinical trial eligibility criteria) using data from a remote database such as clinicaltrials.gov. To do so, the processor may comprise an artificial module intelligence (i.e.: a trained machine learning algorithm) configured to deduce, for each clinical trial, the relevant inclusion and exclusion criteria and compare them to the answers of the medical-team-oriented questions. In other words, to determine if the response meets the clinical trial eligibility criteria, the processor may be configured to select relevant inclusion and exclusion criteria for the or each clinical trial, using a machine learning algorithm trained on a remote database (such as clinicaltrials.gov).

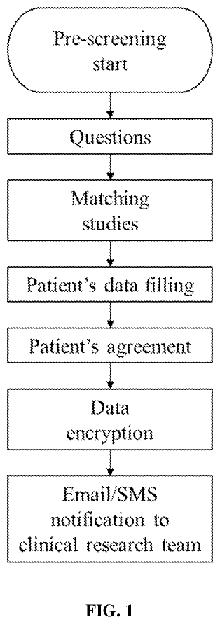

In one embodiment, the system further comprises an encryption module for generating encrypted data encrypted and anonymized patient data, so that a pre-screening result comprising the encrypted and anonymized patient data may further be sent, via the transmission system, to the clinical research team in charge of the selected clinical trial. One example of this embodiment is illustrated in FIG. 1.

In one embodiment, the system is located on a physical or virtual server or hosted on a cloud.

1.2 The Interface

As aforementioned, the interface of the present system is configured to display a list of medical-team-oriented questions and receive the patient's response as collected by the medical team, so as to obtain the real-time answers at the time of pre-screening.

The medical-team-oriented questions reflect at least one, preferably 2 to 15, preferably 5 to 10, clinical trial eligibility criteria.

Non limitative examples of medical-team-oriented questions comprise:
Ongoing insulin?
Associated perianal draining fistula?

In one embodiment, the interface of the present system is configured to display the medical-team-oriented questions as checkbox questions, closed-ended questions, binary questions, multiple-choice questions, and/or numeric questions.

Non limitative examples of medical-team-oriented questions which may be displayed as binary questions comprise:
Ongoing insulin? Yes/No
Associated perianal draining fistula? Yes/No
Pouchitis: Yes/No

A non limitative example of medical-team-oriented questions which may be displayed as multiple-choice question is: 1

Type of treatment: anti-tnf/anti-integrin/anti-il12/23.

In one embodiment, the medical-team-oriented questions are straightforward questions having a number of words ranging from 3 to 35 words, preferably from 4 to 15 words.

In one embodiment, the list of medical-team-oriented questions is configured to be fulfilled in an interval of time comprised between 10 seconds and 5 minutes.

In one embodiment, the list of medical-team-oriented questions is configured to be fulfilled in an interval of time comprised between 1 minute and 3 minutes.

The interface is also configured to display a list of clinical trials. If the processor determines that the patient response meets the eligibility criteria of 1 to 5 clinical trials, a list of said clinical trials is displayed on the interface of the present system.

The processor makes the match and the interface immediately displays, to the member of the medical team connected to the patient, a shortlist of matching studies corresponding to:
clinical trials where recruitment is open and
clinical trials having criteria met by the responses to the pre-screening set of questions, and
possibly, the interface immediately displays further additional features such as study goal, localization etc. . . .

In the present invention, the list of 1-5 clinical trials displayed on the interface and shortlist of matching studies have the same meaning.

Said shortlist of matching studies is directly displayed to the physician in less than a few seconds, preferably less than 10, 5, 3 seconds following the registration of the response to the pre-screening set of questions. The physician selects one clinical trial in the shortlist, i.e. the clinical trial that seems to him the more accurate to the patient's condition.

Displaying a shortlist of matching studies allows to focus the attention over a limited number of clinical trials, thereby easing the analysis of the displayed trials and reducing the time needed to select the more appropriate one. Overall, the clinical trial selection process is improved.

The interface is configured to receive the patient's response collected by the member of the medical team, the selection of one clinical trial from the list, some patient data and the patient agreement.

For instance, the interface may comprise one or more input devices for receiving the response, the clinical trial selection, the patient data and the patient agreement by the member of the medical team. Input devices may comprise a keyboard, a mouse, a touchscreen display.

In one embodiment, the patient data comprise name, surname, telephone number and date of birth of the patient.

In one embodiment, the patient data comprise a patient ID.

In one embodiment, the patient data further comprise an e-mail address; a localization data; place of birth.

In the embodiment of FIG. 1, the interface is referred to as medical-team interface. FIG. 1 shows an embodiment of the invention, where the physician is connected to a patient, for example in a standard consultation, and the patient is susceptible for joining a clinical trial. During the standard consultation, the physician connects to a medical-team interface to start a medical-team-oriented questionnaire of 3 to 15 questions and gets the response of the questions from the patient, in less than 5 minutes. If the responses match the expected responses, i.e. the eligibility, inclusion criteria, and do not show any exclusion criteria, the physician is asked by the interface to get the patient's informed consent and to retrieve patient's data. In one embodiment, all the questions are on one single screen. In one embodiment, some or all of the requested patient's data are directly retrieved from the patient database of the physician. The physician electronically sends the informed consent and the patient's data on the interface to the research team. The interface gets the acknowledgment of the team, and instantly electronically sets an appointment to the patient.

In a second aspect, the present system ensures the pre-screening of a patient with regard to eligibility criteria of at least one clinical trial.

In one embodiment, the interface is configured to display a list of summarized inclusion criteria and exclusion criteria.

In this embodiment, the interface is configured to:
upon selection by the member of the medical team of one clinical trial in the list, display a list of 1 to 5 summarized inclusion criteria and 1 to 5 summarized exclusion criteria for the selected clinical trial;
receive, by the medical team member, the response to the list of summarized criteria;
if the processor determines that the response meets all the inclusion criteria and it does not meet any exclusion criterion, display a message informing that the protocol of the selected clinical trial seems to be compatible with the patient; 1

display a message notifying of the possibility of matching the response with criteria of other clinical trials, while keeping in memory all the already responded criteria; and
display a message asking for (i) collection of patient data that are further encrypted and anonymized in a security layer and (ii) collection of the patient agreement to forward the pre-screening result and anonymized data, to the investigation center.

This embodiment offers the possibility to register in addition to the response previously describe, a second response to the summarized inclusion and exclusion criteria.

Non limitative examples of inclusion criteria and exclusion criteria are:
eGFR >30 ml/min/1.73m²
Endoscopic lesions located at the ileum and/or right colon
Systemic steroids

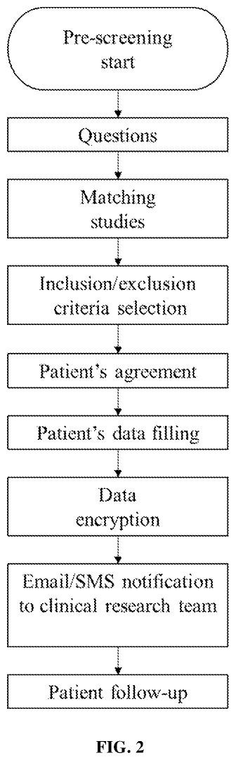

In one embodiment, the summarized inclusion and exclusion criteria are displayed on the interface in form of questions. One example of this embodiment is illustrated in FIG. 2.

FIG. 2 shows an embodiment of the invention, where the physician interacts with a patient susceptible to join a clinical trial and the physician connects to a medical-team interface to start a pre-screening process of the eligibility of the patient to a clinical trial. In one embodiment, the physician is face-to-face with the patient. In one embodiment, the physician is connected to the patient in a virtual appointment (such as for example, a teleconsultation or a phone conversation).

In the embodiment of FIG. 2, the physician connects to the medical-team interface and starts a pre-screening session. The pre-screening session opens a set of questions related to patient's disease information, such as for example:
the identification of the disease (can be skipped if previously assessed)
the disease state, for example active or stable;
the possible complications due to the disease; and/or
the history of treatments.

The pre-screening is based on a limited number of questions ranging from 3 to 15 questions, preferably 5 to 10 questions.

The physician directly requests responses from the patient on the basis of the set of pre-screening questions on the dedicated disease. The information is gathered in less than 5 minutes, preferably in 1 to 3 minutes, and are computed to find out if and what are the clinical trials matching the responses to the pre-screening set of questions.

In the embodiment of FIG. 2, as soon as the selection is made, a new screen window comprising a summarized inclusion and exclusion criteria is displayed on the interface, for instance in form of a second set of questions.

The second set of questions is made from inclusion and exclusion criteria for the selected clinical trial and is clinical trial-dependent. Each clinical trial possesses specific features related to the inclusion and exclusion criteria. The second set of question includes 1 to 5 questions related to inclusion criteria and 1 to 5 questions related to exclusion criteria.

The information used to determine the inclusion or exclusion of patient are treated by a device based on computer unit/system or smartphone or tablet able to interface—read, write, sort, select, compare, extract data—from a database. The physician interacts with the patient to register the response to the second set of question, then selects one clinical trial in the second shortlist appeared on the screen. Then, the physician may gather and fill into a form the patient's data—as first name, last name, date of birth and phone number—and patient's data are encrypted with an encryption algorithm—such as: Blowfish, AES, RC4, DES, RCS, and RC6—and anonymized to result in the clinical trial to be conducted without the risk of contravening a Patient Privacy Regulation. The research team receives instantly a notification (via Email or SMS or any suitable immediate messaging system) related to the patient's entry.

As aforementioned, the interface may also display a message notifying of the possibility of matching the response with criteria of other clinical trials.

Advantageously, this embodiment offers to the medical team member the possibility to verify if the response matches the criteria of clinical trials other than those in the list of clinical trials displayed on the interface.

The possibility to display a first list of clinical trials, and then optionally matching the response with criteria of other clinical trials, allows to perform the selection of the more appropriate clinical trial via a bottom-up approach. This reduces the time needed to select the more appropriate clinical trial while ensuring that the medical team member has the possibility to visualize clinical trials other than those displayed in the first list of clinical trials.

Such rapidity of selection cannot be achieved via top-down approaches, in which a large number of information is displayed at once.

In a third aspect, the system further comprises a patient interface.

In one embodiment, the interface comprises a physician login interface.

In one embodiment, the interface further comprises a patient login interface.

In one embodiment, the interface comprises a patient interface and a medical team interface.

In one embodiment, the patient interface is configured to:
display a list of patient-oriented questions;
register the patient's response to the questions;
displays a list of clinical trials matching the patient's response.
display a message notifying of the possibility of contacting or being contacted by a clinical study coordinator.

This embodiment allows the patient to be put in contact with a coordinator, so that the patient can complete the pre-screening with the help of the coordinator on the medical team interface described hereinabove.

Alternatively, the patient may be allowed to continue the pre-screening on the patient interface. In one embodiment, the patient's response is registered, but no information is sent to the investigation center. In one embodiment, a list of compatible studies and investigation centers may be displayed.

1.2 The Processor

As aforementioned, in the present invention a processor is configured to determine if the response meets the eligibility criteria reflected by the medical-team-oriented questions.

By processor it is meant a processing device, which can for example include a computer, a microprocessor, an integrated circuit, or a programmable logic device (PLD), and which is not restricted to hardware capable of executing software. The processor may also encompass one or more Graphics Processing Units (GPU), whether exploited for computer graphics and image processing or other functions. Additionally, the instructions and/or data enabling to perform associated and/or resulting functionalities may be stored on any processor-readable medium such as, e.g., an integrated circuit, a hard disk, a CD (Compact Disc), an optical disc such as a DVD (Digital Versatile Disc), a RAM (Random-Access Memory) or a ROM (Read-Only Memory). Instructions may be notably stored in hardware, software, firmware or in any combination thereof.

If the response meets the eligibility criteria reflected by the medical-team-oriented questions, the processor determines which clinical trials are linked to said eligibility criteria in the database.

1.3 The Transmission System

The transmission system is configured to send to the clinical research team in charge of the selected clinical trial, a message notifying the pre-screening of the patient.

In one embodiment, the transmission system is further configured to send information related to the medical team member to the clinical research team in charge of the selected clinical trial.

In one embodiment, the transmission system is configured to send to the patient a message notifying the completion of his/her pre-screening.

1.4 Databases and Modules

In one embodiment, the system comprises a database comprising a list of clinical trial eligibility criteria and a list of clinical trials, wherein each clinical trial eligibility criterion is linked to at least one clinical trial, and each clinical trial is linked to at least one eligibility criterion.

In one embodiment, the eligibility criteria and the clinical trials in the database are linked by a many-to-many relationship.

In one embodiment, the system comprises at least one database of straightforward questions and related expected responses.

In one embodiment, the system further comprises an encryption module for generating encrypted data and anonymized patient data.

In one embodiment, the system comprises a storage medium for storing the patient's response.

In one embodiment, the system comprises a message sender module or a connection to a messenger system, to forward the pre-screening results to the investigation center.

In one embodiment, the system comprises at least one patient follow-up module for the follow-up of the patients.

In one embodiment, the system comprises at least one repository comprising the clinical trials open in the investigator center and the full list of eligibility criteria, such as for instance clinical trial inclusion and exclusion criteria. In one embodiment, the repository comprises items accessible to the medical team, and items accessible to investigator team.

In one embodiment, the repository comprises the clinical trial goals or specificities.

In one embodiment, the system comprises at least one users database with a SAAS mode for managing users' access.

In one embodiment, the system comprises at least one patient data form.

In one embodiment, the system comprises at least one encryption, pseudonymization or anonymization software or connection to such software.

In one embodiment, the system comprises a site and patient management module.

The site and patient management module allows to keep tracks of all detected patient and allows to obtain information on the eligibility status available to the relevant site members.

In one embodiment, the site and patient management module allows to send site activity report to sponsors.

The site and patient management module may be configured to allow the interaction of different health professionals in said module.

Advantageously, the site and patient management module facilitates site coordination.

The site and patient management module also allows the system to gather information about, and to manage, various aspects encompassing patient pre-screening, patient management and site management.

The Method

The present invention also relates to a method for pre-screening of a patient with regard to eligibility criteria of at least one clinical trial, preferably during a medical team-patient interaction, comprising:
displaying a list of medical-team-oriented questions reflecting at least one, preferably 2 to 15, preferably 5 to 10, clinical trial eligibility criteria;
registering the patient's response collected by the medical team, so as to obtain the patient's real-time answers at the time of pre-screening;
if the response meets the eligibility criteria of at least 1-5 clinical trials, displaying a list of said clinical trials;
receiving, as input, the selection of one clinical trial in the list of said clinical trials,
collecting patient data and patient agreement; and 1

sending to the clinical research team in charge of the selected clinical trial, a message notifying the pre-screening of the patient.

The present method may be implemented on any computing device, such as for instance a laptop, a tablet, a smartphone.

According to one embodiment, the computing device comprises a network connection enabling remote implementation of the method according to the present invention. According to one embodiment, input data are wirelessly communicated to the processor. According to one embodiment, the interface wirelessly receives the information to be displayed from the processor.

In one embodiment, the whole list of medical-team-oriented questions is displayed on a limited number of screens, preferably on one screen.

In one embodiment, the medical team-patient interaction is a medical face-to-face or virtual appointment.

In one embodiment, the method of the present invention is preceded by:
logging in into a patient interface displaying a questionnaire containing patient-oriented questions;
registering of the response to the patient-oriented questions;
comparing the response to the eligibility criteria of clinical trials stored in a database;
if the response meets the eligibility criteria of the clinical trials, displaying a list of said clinical trials.

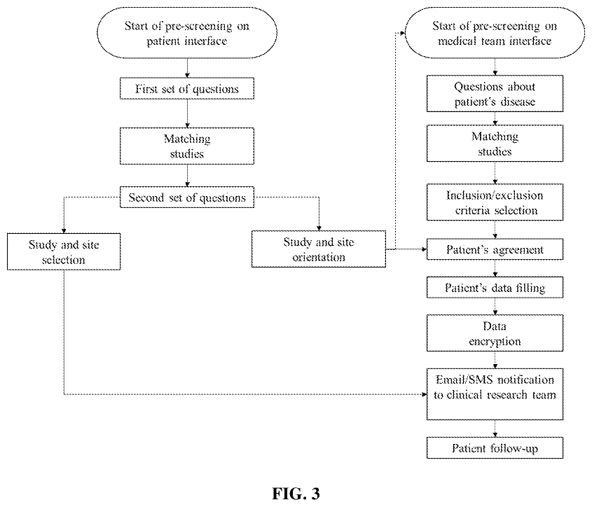

In this embodiment, the patient may connect to a patient interface, and answers a patient's questionnaire containing patient-oriented questions only. Upon registration of the response, the patient interface displays a number of matching studies. From that point, either a coordinator contacts the patient, and the patient accepts to continue with the help of the coordinator on a second questionnaire and personal data get from the system information on the investigation centers and/or studies and goes to investigation center, where he is helped to continue the prescreening process on the medical team interface. Alternatively, the patient continues and records his/her response, but no information is sent to the investigation center. The patient will have access to a list of compatible studies and investigation centers to contact. One example of this embodiment where the process begins with a patient questionnaire, with patient-oriented questions, on a patient interface is illustrated in FIG. 3.

The coordinator can be a physician, or a specialist of clinical trials, especially those present on the system. His role is to further assess if the patient may be successfully pre-screened.

When the patient accepts to be contacted by a coordinator, they discuss the possible prescreening of the patient, then, the pre-screening results shall be sent to the investigation center.

In one embodiment, the data of the patient prescreening are aggregated for further uses, such as analytics, improvement processes, feasibility studies or data trading.

In one embodiment, some or all of the data of the pre-screenings, without any personal data, are aggregated and made accessible, for information to Sponsor (i.e. Sponsor of the clinical trial for which the aggregated pre-screenings was carried out).

In one embodiment, some aggregated pre-screenings are made accessible to a contract research organization, for research needs.

In one embodiment, some aggregated pre-screenings are made accessible to the Investigation Center.

In one embodiment, aggregated pre-screenings are used for designing possible corrective actions in order to improve pre-screening and increase patient recruitment.

In one embodiment, some or all of the data of the pre-screenings, from various investigation centers are aggregated, concatenated and compared with the criteria of a future study of a sponsor in order to recommend/select the most appropriate centers to the sponsor (feasibility study).

In a fourth aspect of the present invention, some or all of the data of the pre-screenings are aggregated, concatenated, in order to define and analyze patient populations or define patient population patterns, for example but not limitatively to analyze patient that are enrolled or dismissed, after having pre-screened, or to analyze if and how patients actually meet or actually do not meet some of the criteria. An advantage of this, is to enhance criteria compatibility to patient populations.

The Computer Program and Storage Medium

The present method also relates to a computer program comprising program code instructions which, when executed by a computer, cause the computer to carry out the method according to anyone of the embodiments described hereabove.

Computer program code for carrying out operations for aspects of the present invention may be written in any combination of one or more programming languages, including an object-oriented programming language such as Java, Python, Ruby, PHP, C++ or the like and conventional procedural programming languages, such as the “C” programming language or similar programming languages. The computer program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Computer programs implementing the method of the present embodiments can commonly be distributed to users on a distribution computer-readable storage medium such as, but not limited to, an SD card, an external storage device, a microchip, a flash memory device and a portable hard drive. From the distribution medium, the computer programs can be copied to a hard disk or a similar intermediate storage medium. The computer programs can be run by loading the computer instructions either from their distribution medium or their intermediate storage medium into the execution memory of the computer, configuring the computer to act in accordance with the method of this invention. All these operations are well- known to those skilled in the art of computer systems.

The present method also relates a non-volatile computer storage medium comprising instructions which, when executed by a computer, cause the computer to carry out the method according to anyone of the embodiments described hereabove.

While various embodiments have been described and illustrated, the detailed description is not to be construed as being limited hereto. Various modifications can be made to the embodiments by those skilled in the art without departing from the true spirit and scope of the disclosure as defined by the claims.

### EXAMPLE

The present invention is further illustrated by the following example, evaluating the number of patients enrolled and the number of patients randomized in two phase III clinical trials evaluating the efficacy and safety of etrolizumab in ulcerative colitis and Crohn' s disease in sites equipped or non-equipped with the present invention.

Materials and Methods

134 sites having being opened for more than 6 months were included in the present study. 1-way analysis of variance followed by post hoc Tukey and Mann-Whitney tests was performed to compare the number of patients pre-screened, screened, and randomized, in the sites utilizing the present invention (columns A) and in the sites non-equipped with the present invention (columns B), with p-value <0.05 considered significant.

The pre-screening efficiency was calculated as the mean number of screened patients per site per month, divided by mean number of pre-screened patients per site per month.

The recruitment rate was calculated as the mean number of randomized patients per site per month, divided by mean number of pre-screened patients per site per month.

Results

The mean number of screened and randomized patients per month per site in both studies is represented in the Table 1.

TABLE 1

| All clinical trials combined | A | B | p-value |
|---|---|---|---|
| Screened patients | 7.55 | 3.05 | <0.001 |
| Randomized patients | 3.79 | 1.27 | <0.001 |

The mean number of screened and randomized patients per month per site in the clinical trial “HICKORY” and in the clinical trial “BERGAMOT” is represented in Table 2 and Table 3, respectively.

TABLE 2

| Clinical trial “HICKORY” | A | B | p-value |
|---|---|---|---|
| Screened patients | 9.17 | 3.14 | <0.001 |
| Randomized patients | 5.17 | 1.28 | <0.001 |

TABLE 3

| Clinical trial “BERGAMOT” | A | B | p-value |
|---|---|---|---|
| Screened patients | 5.94 | 2.97 | =0.003 |
| Randomized patients | 2.41 | 1.26 | <0.001 |

The pre-screening efficiency and recruitment rate obtained with the present invention are represented in Table 4.

| | Pre- | | Pre- | Random- | Recruit- |
|---|---|---|---|---|---|
| | screened | Screened | screening | ized | ment |
| | patients | patients | efficiency | patients | rate |
| HICKORY | 13.9 | 9.17 | 0.66 | 5.17 | 0.37 |
| BERGAMOT | 15.9 | 5.94 | 0.37 | 2.41 | 0.15 |

These results show that the mean number of screened and randomized patients is higher when patients are pre-screened with the present invention, when compared to the mean number of screened and randomized patients in case the pre-screening is performed without the system of the invention.

1. A system for pre-screening of a patient with regard to eligibility criteria of at least one, preferably 2-5 clinical trials, preferably during a medical team-patient interaction, the system comprising an interface, a processor, and a transmission system, wherein the interface is configured to:
display a list of medical-team-oriented questions reflecting at least one, preferably 2 to 15, preferably 5 to 10, clinical trial eligibility criteria of at least one, preferably 2-5 clinical trials,
receive the patient's response, the response being collected by the medical team, so that they are real-time answers at the time of pre-screening,
if the response meets the eligibility criteria of at least 1-5 clinical trials, displays a list of said clinical trials,
receive a selection of one clinical trial by the physician in the list by a member of the medical team, receive patient data and receive agreement of the patient;
wherein the medical-team-oriented questions are straightforward questions having a number of words ranging from 3 to 35 words, preferably from 4 to 15 words and wherein the processor is configured to determine if the response meets the clinical trial eligibility criteria of at least one clinical trial; and the transmission system is configured to sends to the clinical research team in charge of the selected clinical trial, a message notifying the pre-screening of the patient.

2. The system according to claim 1, wherein the interface is further configured to record the medical-team-oriented questions in the form of a decision tree.

3. The system according to claim 1, to determine if the response meets the clinical trial eligibility criteria, the processor is configured to select relevant inclusion and exclusion criteria for the or each clinical trial, using a machine learning algorithm trained on a remote database.

4. The system according to claim 1, wherein the interface is configured to display the medical-team-oriented questions are displayed as checkbox questions, closed-ended questions, binary questions, multiple-choice questions and/or numeric questions.

5. The system according to claim 1, wherein the list of medical-team-oriented questions is drawn to be fulfilled in 10 seconds to 5 minutes, preferably 1 to 3 minutes.

6. The system according to claim 1, wherein the patient data comprises name, surname, telephone number and date of birth of the patient.

7. The system according to claim 5, wherein the patient data further comprises an e-mail address; a localization data; place of birth.

8. The system according to claim 1, further comprising a storage medium for storing the patient's response.

9. The system according to claim 1, further comprising a database comprising a list of clinical trial eligibility criteria and a list of clinical trials, wherein each clinical trial eligibility criterion is linked to at least one clinical trial, and each clinical trial is linked to a clinical research team.

10. The system according to claim 1, further comprising an encryption module for generating encrypted data encrypted and anonymized patient data.

11. The system according to claim 1, wherein the interface is further configured to:
upon selection of one clinical trial in the list, display a list of 1-5 summarized inclusion criteria and 1-5 summarized exclusion criteria for the selected clinical trial;
receive the response to the list of summarized inclusion and exclusion criteria,
if the response meets the inclusion criteria and none of the exclusion criteria, display a message informing the member of the medical team that the protocol of the clinical trial seems to be compatible with the patient,
display a message notifying of the possibility of matching with criteria of other clinical trials,
display a message asking for (i) collection of patient data and (ii) collection of the patient agreement; and
wherein the transmission system is configured to send a pre-screening result comprising the anonymized data, to the clinical research team.

12. The system according to claim 1, wherein the transmission system is further configured to send information related to the medical team member to the clinical research team.

13. The system according to claim 1, said system being located on a physical or virtual server or hosted on a cloud.

14. The system according to claim 1, further comprising:
at least one message generator module,
at least one patient follow-up module for the follow-up of the patients,
at least one repository with the clinical trials open in the investigator center with the full list of eligibility criteria including non limitatively inclusion/exclusion criteria, said repository comprising a set of items accessible to the medical team, and a set of items accessible to investigator team,
at least one piece of key information on the clinical trial goals or specificities,
at least one database of straightforward questions and related responses,
at least one users database with a SAAS mode for managing users' access,
at least one patient data form,
at least one encryption, pseudonymization or anonymization software or connection to such software, and/or
at least one message sender or connection to any messenger system, to forward the pre-screening results to the investigation center.

15. A computer-implemented method for pre-screening of a patient with regard to eligibility criteria of at least one clinical trial, preferably during a medical team-patient interaction, comprising:
displaying a list of medical-team-oriented questions reflecting at least one, preferably 2 to 15, preferably 5 to 10, clinical trial eligibility criteria;
receiving, as input, the patient's response collected by a medical team member, so as to obtain the patient's real-time answers at the time of pre-screening;
if the response meets the eligibility criteria of at least 1-5 clinical trials, displaying a list of said clinical trials;
receiving, as input, the selection of one clinical trial in the list of said clinical trials,
collecting patient data and patient agreement;
sending to the clinical research team in charge of the selected clinical trial, a message notifying the pre-screening of the patient.

16. The method according to claim 14, wherein the list of medical-team-oriented questions is displayed on a limited number of screens, preferably on one screen.

17. The method according to claim 14, wherein the medical team-patient interaction is a medical face-to-face or virtual appointment.

18. The method according to claim 14, further preceded by:
logging into a patient interface displaying a questionnaire containing patient-oriented questions reflecting clinical trial eligibility criteria;
receiving the response to the patient-oriented questions;
if the response meets the eligibility criteria of at least one clinical trial, displaying the at least one clinical trial.

19. A computer program comprising program code instructions which, when executed by a computer, cause the computer to carry out the method according to claim 15.

20. A non-volatile computer storage medium comprising instructions which, when executed by a computer, cause the computer to carry out the method according to claim 15.
